(19) United States
(12) Patent Application Publication
(10) Pub. No.: US 2018/0207435 A1
(43) Pub. Date: Jul. 26, 2018

(54) MOBILE DEFIBRILLATOR FOR USE WITH PERSONAL MULTIFUNCTION DEVICE AND METHODS OF USE

(71) Applicant: Smart Savior, Spring, TX (US)

(72) Inventor: Paul Michael Yetter, Spring, TX (US)

(21) Appl. No.: 15/874,213

(22) Filed: Jan. 18, 2018

Related U.S. Application Data
(60) Provisional application No. 62/449,259, filed on Jan. 23, 2017.

(51) Int. Cl. A61N 1/39; A61N 1/04; A61N 1/08

(52) U.S. Cl. CPC A61N 1/3904; A61N 1/046; A61N 1/3993; A61N 1/3925; A61N 1/08; A61N 1/0492

(57) ABSTRACT

A portable defibrillator system includes a personal multifunction device and a portable defibrillator device for delivering a dose of electric current to a heart for ending the dysrhythmia and allowing the heart to re-establish a normal sinus rhythm. A portable defibrillator case contains defibrillation circuitry supporting the generation and transmission of a dose of electric current. A set of charging pads electrically connect to the portable defibrillator case for communication of the dose of electric current and sensing from the patient responses to said electric current. A first set of communication circuitry communicates between the defibrillation circuitry and a personal multifunction device. A second set of communication circuitry communicates between the defibrillation circuitry and the set of charging pads. The portable defibrillator case, set of charging pads, and first and second sets of communication circuitry are of a size similar to the size of the personal multifunction device.

### CROSS REFERENCE TO RELATED APPLICATIONS

This application further claims the benefit of the following provisional application which is hereby expressly incorporated by reference:

62/449,259 entitled “MOBILE DEFIBRILLATOR,” filed on Jan. 23, 2017 with Attorney Docket No. SMAR001USP.

### FIELD OF THE PRESENT DISCLOSURE

The inventive subject matter generally relates to a handheld portable defibrillator. In particular, the inventive subject matter relates to a handheld portable defibrillator that connects to a personal multifunction device (“PED”), such as a smart phone.

### BACKGROUND OF THE PRESENT DISCLOSURE

Cardiac arrest is a sudden stop in blood flow due to the failure of the heart to pump blood. Many instances of cardiac arrest occur outside of hospitals or far away from medical attention. In the United States, over 300,000 people experience a cardiac arrest outside of the hospital. When blood is not flowing to the brain, every second counts. For every minute of the heart not pumping, the chances of survival for the sufferer decrease by 8-10%.

Defibrillation is a treatment for life-threatening cardiac dysrhythmias, specifically ventricular fibrillation (VF) and non-perfusing ventricular tachycardia (VT). A defibrillator delivers a dose of electric current (often called a counter shock) to the heart. This depolarizes a large amount of the heart muscle, ending the dysrhythmia. Subsequently, the body's natural pacemaker in the sinoatrial node of the heart may re-establish normal sinus rhythm.

Defibrillators can be external, transvenous, or implanted (implantable cardioverter-defibrillator), depending on the type of device used or needed. Some external units, known as automated external defibrillators (AEDs), automate the diagnosis of treatable rhythms, meaning that lay responders or bystanders may use them successfully with little or no training.

The exact mechanism of defibrillation is not well understood. One theory is that successful defibrillation affects a critical mass of the heart, resulting in insufficient remaining heart muscle to continue the arrhythmia. Recent mathematical models of defibrillation are providing new insight into how cardiac tissue responds to a strong electrical shock.

Manual external defibrillators require the expertise of a healthcare professional. They are used in conjunction with an electrocardiogram, which can be separate or built-in. A healthcare provider may first diagnose the cardiac rhythm and then manually determine the voltage and timing for the electrical shock. These units are primarily found in hospitals and on some ambulances. In the United States, many advanced EMTs and all paramedics are trained to recognize lethal arrhythmias and deliver appropriate electrical therapy with a manual defibrillator when appropriate.

An automated external defibrillator or (AED) contain technology for analysis of heart rhythms. As a result, it does not require a trained health provider to determine whether a rhythm is shockable. By making these units publicly available, AEDs have improved outcomes for sudden out-of-hospital cardiac arrests. Trained health professionals have more limited use for AEDs than manual external defibrillators. An automated external defibrillator ready for use. Pads are pre-connected. This model is a semi-automatic due to the presence of a shock button. As early defibrillation can significantly improve VF outcomes, AEDs have become publicly available in many easily accessible areas. AEDs have been incorporated into the algorithm for basic life support (BLS).

A breakthrough was the introduction of portable defibrillators used out of the hospital. Today portable defibrillators are among the many very important tools carried by ambulances. They are the only proven way to resuscitate a person who has had a cardiac arrest unwitnessed by Emergency Medical Services (EMS) who is still in persistent ventricular fibrillation or ventricular tachycardia at the arrival of pre-hospital providers. Yet, these portable defibrillators are not easy to carry around and can only be used by EMS, in most cases.

There is an increase in availability of defibrillators; however, they are not always nearby. The defibrillators also suffer from a weakness that they are only checked if they work properly when there is an emergency. If the battery is too low to supply enough energy to the patient, or the device fails, there is often no other option but to wait for help to arrive. Help is often far away, taking on average eight (8) minutes to reach a victim. By this time, the patient has a low chance of survival, and has suffered severe brain loss.

Accordingly, a need exists for a more portable and more reliable defibrillator.

### BRIEF SUMMARY OF THE PRESENT DISCLOSURE

This summary presents integral concepts in a simplified form as a prelude to the more detailed disclosure that is presented herein. Unless otherwise defined, all terms (including technical and scientific terms) used herein have the same meaning as commonly understood by one of ordinary skill in the art to which this disclosure belongs. It may be further understood that terms, such as those defined in commonly used dictionaries, should be interpreted as having a meaning that is consistent with their meaning in the context of the relevant art and the present disclosure, and may not be interpreted in an idealized or overly formal sense unless expressly so defined herein.

In one aspect of the inventive subject matter, a mobile defibrillator is disclosed. The embodiment provides for a portable defibrillation system that can easily be carried in a pocket or purse. This ease of portability allows people at high risk of cardiac arrest to have a defibrillator nearby. This proximity of a defibrillator will lead to reduced treatment times after a cardiac arrest occurs, and ultimately it will increase the odds of survival and reduce the damaging effects of lack of blood flow.

In another aspect of the present disclosure, here is provided a portable defibrillator device for use with a mobile phone or personal multifunction device for delivering a dose of electric current or counter shock to a heart. The disclosed subject matter provides the electric shock for depolarizing a large amount of the heart muscle, thereby ending dysrhythmia and in response thereto allowing the heart to re-establish a normal sinus rhythm. The disclosure includes a portable defibrillator case for containing defibrillation circuitry supporting the generation and transmission of a dose of electric current or counter shock. A set of charging pads electrically connect to the portable defibrillator case for communication of the dose of electric current or counter shock to a patient and sensing from the patient responses to said electric current or counter shock. A first set of communication circuitry associated with said portable defibrillator case for communicating between said defibrillation circuitry and said mobile phone or personal multifunction device. A second set of communication circuitry associated with said portable defibrillator case for communicating between said defibrillation circuitry and said set of charging pads. The portable defibrillator case, the set of charging pads, and the first and second sets of communication circuitry are of a size essentially similar to the size of the mobile phone or personal multifunction device.

The portable defibrillator device includes defibrillation circuitry having a charging circuit for generating and transmitting from said portable defibrillator case a defibrillation charge of electricity of sufficient charge and current for depolarizing at least a portion of a heart muscle for ending dysrhythmia in the heart muscle. A set of charging pad circuits receive the defibrillation charge and apply said defibrillation charge to the torso of a patient in the proximity of the heart and a set of charging pads comprising charging circuitry for applying a dose of electric current or counter shock to a heart in response to energization from said charging pad circuits.

The portable defibrillator further includes an EKC sensing circuit for generating, sending and receiving from said portable defibrillator case EKG sensing signals for monitoring the heart during the administration of said defibrillation charge. The set of charging pad circuits further comprise EKG sensing circuitry for sending and receiving EKG heart monitoring signals between said set of charging pads and said mobile phone or personal multifunction device. The set of charging pads comprise EKG signal circuitry for generating EKG sensing signals for transmission to said EKG sensing circuitry. The portable defibrillator device circuitry further includes a resistance sensing circuit for generating, sending and receiving from said portable defibrillator case a plurality of resistance sensing signals for monitoring the heart and operation of said EKG sensing circuit and said set of charging pads during the administration of said defibrillation charge. The resistance sensing circuit further comprises resistance reporting for reporting a plurality of resistance sensing signals for assessing and reporting the operational status of said portable defibrillator device.

The portable defibrillator device further includes interface circuitry for interfacing the portable defibrillator device with a mobile phone or personal multifunction device. This supports coordinating the operations and the portable defibrillator device with the mobile phone or personal multifunction device to provide controlled delivery of a dose of electric current or counter shock to a heart and continuous monitoring of the status of the heart in response to said controlled delivery.

The portable defibrillator circuitry includes a wireless communication circuit for communicating wireless communications with a plurality of communications circuits providing services related to the operation of said portable defibrillation device. A set of operational and sensing instructions for storing on a memory circuit associated with said mobile phone or personal multifunction device, said set of operational and sensing instructions for controlling the operation of said portable defibrillator device using the mobile phone or personal multifunction device.

According to one aspect of the present disclosure, there is here shown a method of verifying the defibrillator is functioning and ready for immediate use.

Descriptions of certain illustrative aspects are described herein in connection with the annexed FIGUREs. These aspects are indicative of various non-limiting ways in which the disclosed subject matter may be utilized, all of which are intended to be within the scope of the disclosed subject matter. Other advantages, emerging properties, and features may become apparent from the following detailed disclosure when considered in conjunction with the associated FIGUREs that are also within the scope of the disclosure.

### BRIEF DESCRIPTION OF THE DRAWINGS

The novel features believed characteristic of the disclosed subject matter may be set forth in any claims that are filed later. The disclosed subject matter itself, however, as well as a preferred mode of use, further objectives, and advantages thereof, may best be understood by reference to the following detailed description of an illustrative embodiment when read in conjunction with the accompanying drawings, wherein:

FIGS. 1A, 1B, and 1C illustrate various uses of the portable defibrillator of the present disclosure;

FIGS. 2 and 3 depict various aspects of the portable defibrillator according to an embodiment of the disclosed subject matter;

FIGS. 4A and 4B show aspects of the portable defibrillator in operation with personal multifunction device; and

FIGS. 5 and 6 show functional aspects of the portable defibrillator in operation with a personal multifunction device;

FIG. 7 shows how the portable defibrillator of the present disclosure may serve a patient needing medical care;

FIG. 8 depicts an aspect of the portable defibrillator of the present disclosure in the event of malfunction;

FIG. 9 shows how the personal multifunction device may provide instructions for using the portable defibrillator of the present disclosure;

FIG. 10 illustrates how the personal multifunction device presents heart rate and related information;

FIGS. 11 and 12 show automatic and assisted operation of the portable defibrillator of the present disclosure;

FIG. 13 displays an interface a physician or skilled medical professional may use to manually operate the portable defibrillator of the present disclosure;

FIG. 14 portrays the transmission of patient vital statistics from the presently disclosed portable defibrillation system to a medical facility;

FIGS. 15 and 16 show system reports available through the personal defibrillator system of the present disclosure;

FIGS. 17 and 18 show an exemplary personal multifunction device for use with the portable defibrillator of the present disclosure;

FIGS. 19 and 20 present functional aspects of the system of the present disclosure.

### DETAILED DESCRIPTION

Reference now should be made to the drawings, in which the same reference numbers are used throughout the different FIGUREs to designate the same components.

The terminology used herein is for the purpose of describing particular embodiments only and is not intended to be limiting. As used herein, the singular forms “a”, “an”, and “the” are intended to include the plural forms as well, unless the context clearly indicates otherwise. It may be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although described with reference to personal computers and the Internet, one skilled in the art could apply the principles discussed herein to any computing or mobile computing environment. Further, one skilled in the art could apply the principles discussed herein to communication mediums beyond the Internet.

It will be appreciated that for simplicity and clarity of illustration, where considered appropriate, reference numerals may be repeated among the FIGUREs to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, well-known methods, procedures and components have not been described in detail so as not to obscure the implementations described herein. Also, the description is not to be considered as limiting the scope of the implementations described herein.

In the following detailed description, reference is made to the accompanying drawings that form a part hereof, and in which is shown by way of illustration specific implementations which may be practiced. These implementations are described in sufficient detail to enable those skilled in the art to practice the implementations, and it is to be understood that other implementations may be utilized and that logical, mechanical, electrical and other changes may be made without departing from the scope of the implementations. The following detailed description is, therefore, not to be taken in a limiting sense.

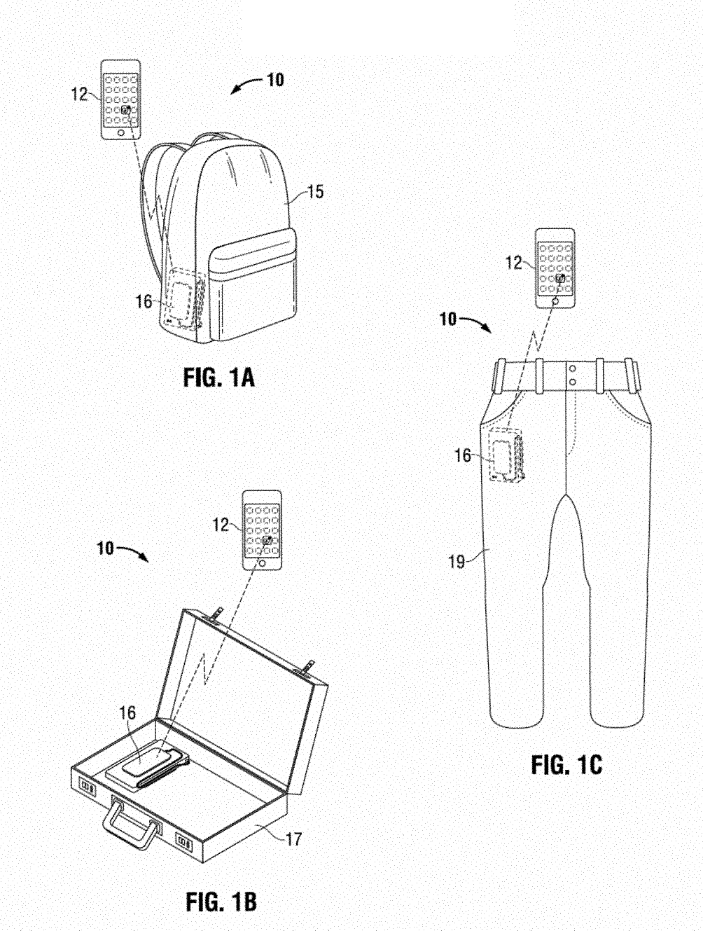

FIGS. 1A, 1B, and 1C depict a portable defibrillation system 10 that includes a personal multifunction device 12 with which the portable defibrillation system 10 of the present disclosure may operate. Personal multifunction device 12 may include a defibrillator control app 12, as illustrated by the defibrillation icon. Selectable control of portable defibrillator device 16 of the present disclosure occurs through the use of defibrillation control app 12.

Referring to FIGS. 1A through 1C, the point becomes clear that portable defibrillator device 16 may comfortably fit within a backpack 15 (FIG. 1A), a brief case 17 (FIG. 1B and/or even a pair of slacks 19 (FIG. 1C). This portable device provides a handy way of meeting the medical requirements for defibrillation when the situation calls for that ability.

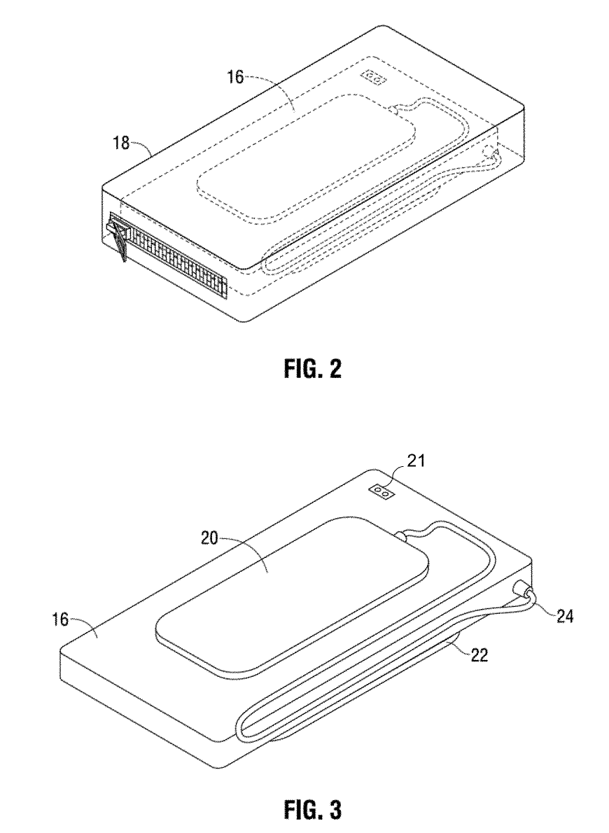

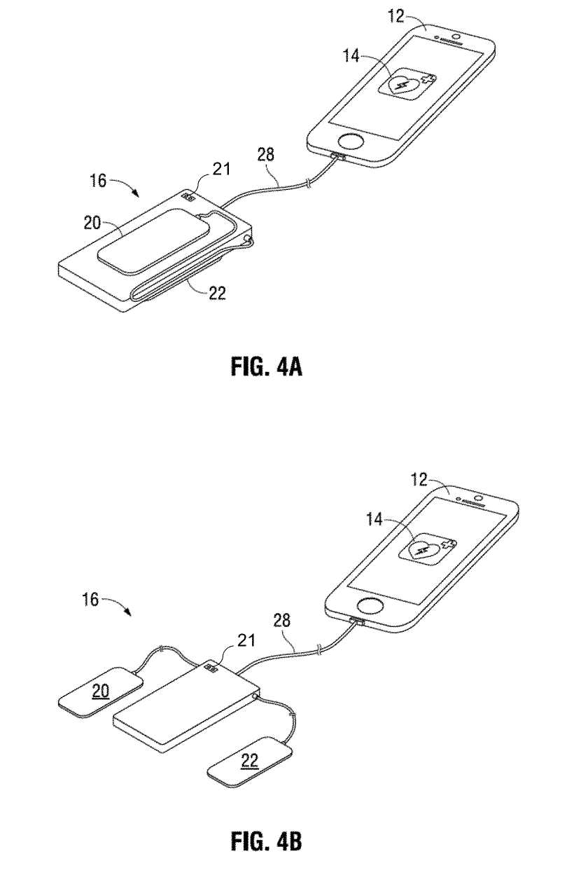

FIGS. 2 and 3 depict various aspects of the portable defibrillator device 16 according to an embodiment of the disclosed subject matter. Portable defibrillator device 16 fits comfortably within carrying case 18. The device is similar in size to that of a portable multifunction device, such as a mobile phone 12 of FIGS. 1A through 1C. FIG. 3 shows that portable defibrillator device 16 includes charging pads 20 and 22, which connect via wires 24 and 26 (see, FIG. 4B), for connecting to the hardware and software within portable defibrillation system 10. In addition, portable defibrillator device 16 includes status LEDs 21 which show the operational status of the device and can be programmed to indicate a variety of different operational conditions, including charging, low power, circuit failure and other status according to different long and short flashing or other variations.

FIGS. 4A and 4B show aspects of the portable defibrillator in operation with personal multifunction device. Portable defibrillator device 16 connects to personal multifunction device 12 via connection cable 28. When connected and communicating, personal multifunction device 12 and portable defibrillator device 16 cooperate to provide a powerful and reliable system for patient defibrillation. Portable defibrillator device 16 includes necessary components required for storing energy, discharging energy, recording information, sending and receiving information, and connecting with a personal multifunction device.

The attached personal multifunction device 12 may comprise multiple circuits useful for gathering, receiving, and sending information. The attached personal multifunction device may be a mobile phone or other personal multifunction device capable of performing cellular or wireless communications, as well as a wide variety of processing functions and algorithms for carrying out the functions and features here described relating to the necessary functionality for interfacing and controlling portable defibrillator device 16. In one embodiment, a personal multifunction app may operate to enable the portable defibrillation system 10 to record heart and other biometric data from a person that is in contact with portable defibrillator device 16. The defibrillator app 14 may then process the biometric data or send it over a wireless internet connection or a cellular connection to a computer that is accessible by the owner of the device or a medical service. Some embodiments may be configured to transmit the information to the cardiac arrest patients doctor or medical facility where they are being treated.

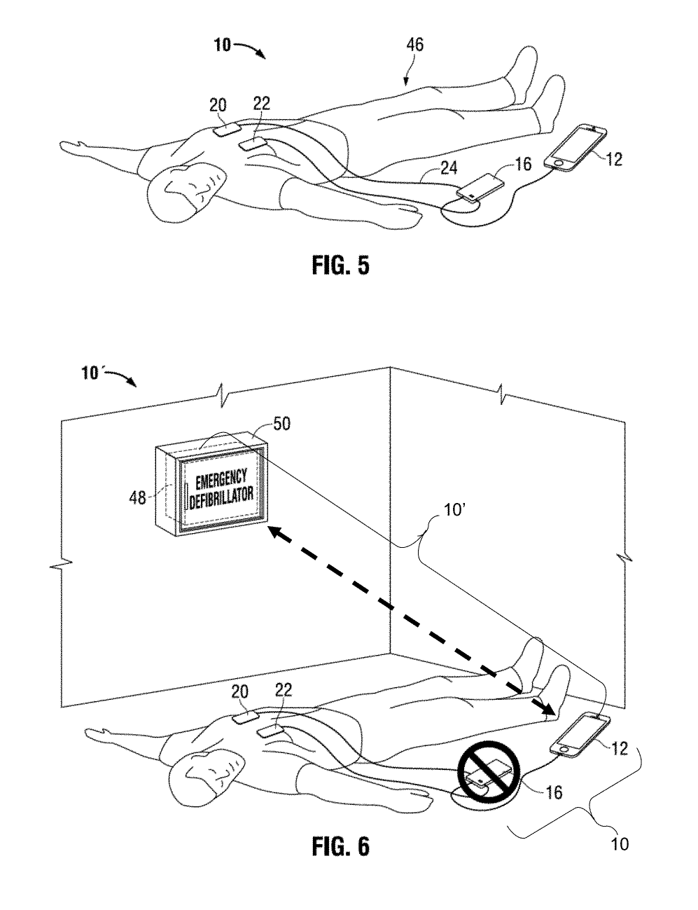

FIGS. 5 and 6 show functional components within portable defibrillator device 16. Thus, referring to FIG. 5, battery 3026 provides power to charging circuit 32, EKG sensor 34, resistance sensor circuit 36, wireless/Bluetooth circuit 38, phone/interface circuit 40, and QA/QC circuit 42. FIG. 6 illustrates the configuration of portable defibrillator device 16 associated, via cable 28 with personal multifunction device 12. Control of portable defibrillator device 16 occurs via the personal multifunction device 16 defibrillator application which presents screen 44 to the user.

Battery 30 may be single use, replaceable, or rechargeable and preferably has enough power to accommodate repeated shock levels as recommended and known by persons having skill in the art. The portable defibrillation system 10 preferably comprises at least two electrodes as required to create a path for the voltage to cross the heart. The one or more capacitors 30 enable the rapid discharge of voltage through the electrical system, including the electrodes. The first charging pad 20 and a second charging pad 22 are electrically connected to battery 30 and capacitors of charging circuit 32 to enable the transfer of voltage to the patient.

The phone/interface circuit 40 may be configured to enable communication with personal multifunction device 12, through a wired or wireless method. The phone/interface circuit 40 may also be configured to amplify the signals received from the patient connected by via the electrodes. Optionally, portable de may further comprise wireless/Bluetooth circuit 38 enabling the portable defibrillator device 16 to wirelessly communicate with a nearby personal multifunction device 12 such as a personal multifunction device with a personal multifunction app that may control the portable defibrillator device 16.

The application may also enable the portable defibrillation system 10 to check the status of the battery 30 and other components of the portable defibrillator device 16 to verify that all components are able to operate and will function in the event that it is required. This process may be directed by defibrillator application 14 either through the cable 28 or through a wireless connection without any intervention required by the user.

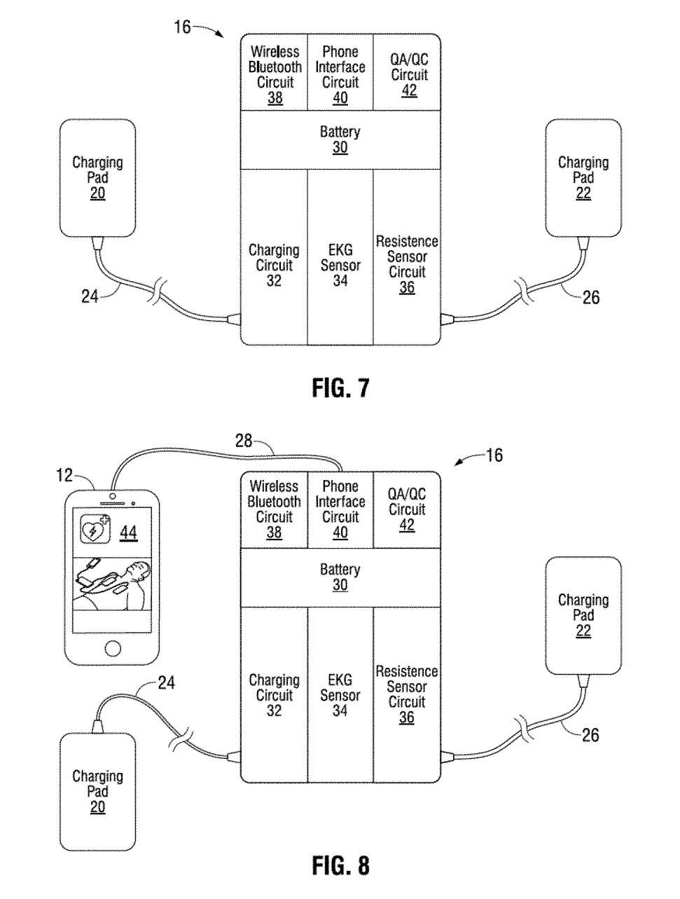

FIG. 7 shows how portable defibrillation system 10 may serve a patient 46 needing medical care include defibrillation shock. Thus, when patient 46 requires emergency defibrillation, portable defibrillator device 16 may be connected to personal multifunction device 12. Based on the connection of charging pad 20 through charging wire 24 and charging pad 22 via charging wire 24 to portable defibrillator device 16, sufficient electrical charge and control of the defibrillation process occurs for patient 46 to receive a dose of electric current or counter shock to the heart, for depolarizing a large amount of the heart muscle, thereby ending the dysrhythmia and in response thereto allowing the heart to re-establish a normal sinus rhythm.

FIG. 8 depicts an aspect of the portable defibrillator of the present disclosure in the event of malfunction. A highly attractive aspect of the defibrillation system of the present disclosure is the ability of personal multifunction device 12 to sense a malfunction with portable defibrillation device 16 and respond. Normally, the combination of personal multifunction device 12 and portable defibrillation device 12 form defibrillation system 10. The response that defibrillation application 14 may provide is to cease communicating with a malfunctioning portable defibrillator device and seek to identify a nearby alternate portable defibrillator tribulation device 48. Thus, as FIG. 8 portrays, if portable defibrillation device 16 malfunctions, personal multifunction device 12 may search to find that alternate portable defibrillation device 48, which happens to be nearby. In this example, portable defibrillation device 48 happens to be with in first aid kit 50. The new combination of personal multifunction device 12 and alternate portable defibrillation device 48 may be considered alternate defibrillation system 10′. First aid kit 50 may be either a portable first aid kit or it may be a wall-mounted first aid kit. In any event, portable multifunction device 12 using defibrillation application 14 may provide redundant reliability in a variety of emergency conditions.

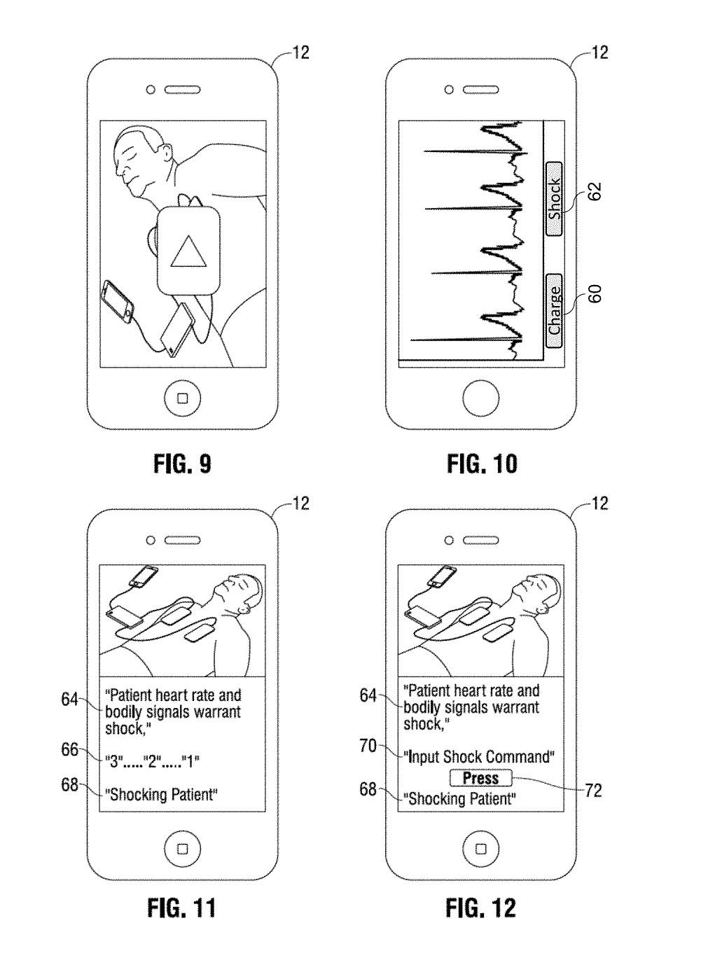

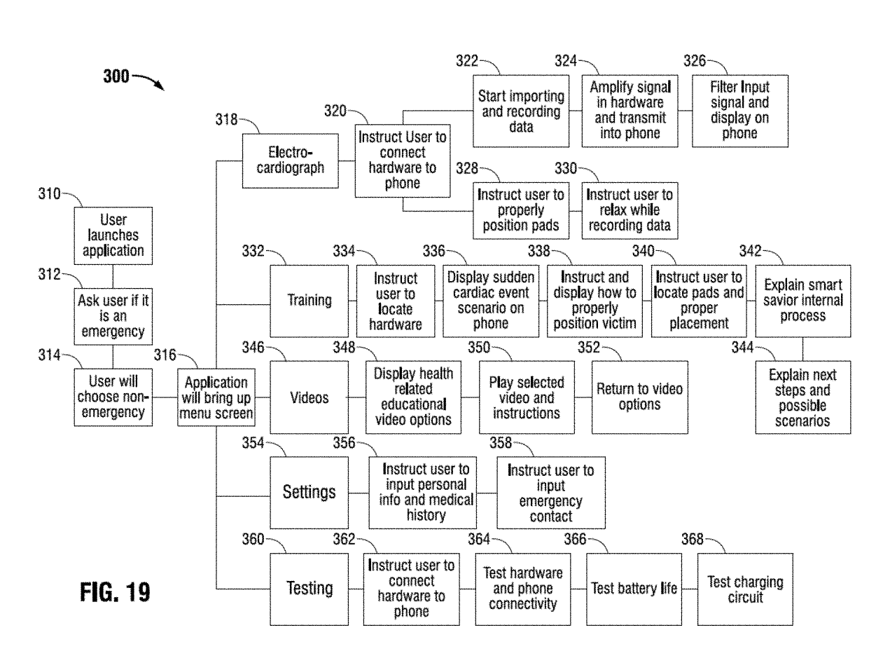

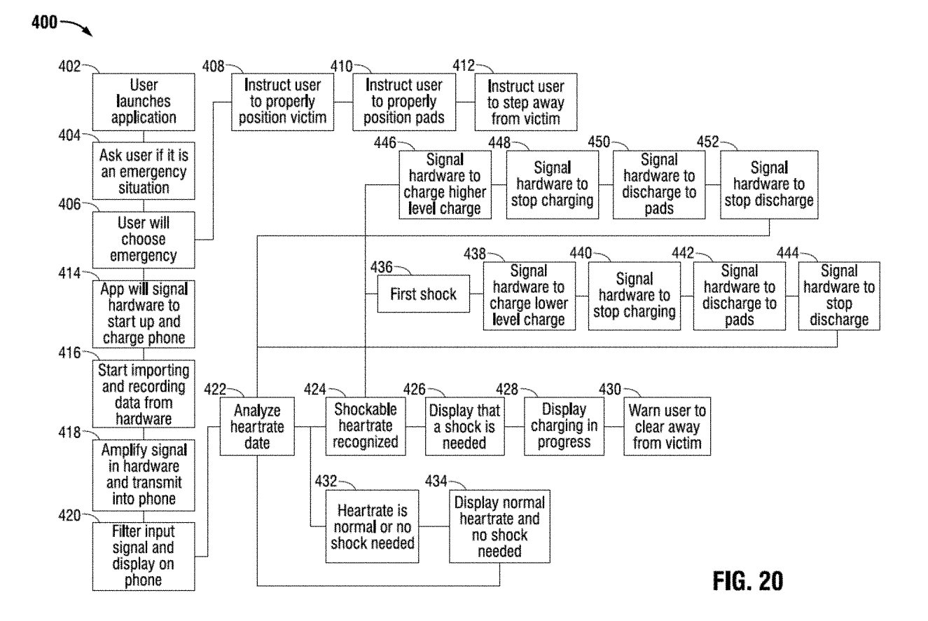

FIG. 9 shows how the personal multifunction device may provide instructions for using portable defibrillation system 10 of the present disclosure. Defibrillation application 14 will be discussed now in FIGS. 9 through 16 and in FIGS. 19 and 20. For instance, FIG. 9 depicts that upon activating defibrillation application 14, the user may be presented with an instructional video as shown in screen 52 to illustrate how to use portable defibrillation system 10. By clicking video icon 54, the instructional video begins to educate the user in how to operate personal defibrillation system 10.

FIG. 10 illustrates how the personal multifunction device presents heart rate information and control portable defibrillation device 16. In addition, FIG. 10 shows heart rate monitor screen 56 for indicating the sensing of a patient 46 heart rate when charging pads 20 and 22 are applied. Moreover, from heart rate monitor screen 56, charging pads 20 and 22 may be charged, as button 60 indicates, and operated to shock patient 46, as button 62 indicates.

FIGS. 11 and 12 depict automated and semi-automated or guided defibrillation that defibrillation application 14 makes possible. Referring to FIG. 11, automated defibrillation is indicated where indication 64 states that “Patient heart rate and bodily signals warrant shock” as an indication that a charging sequence will begin. Following indication 64, a countdown (“3,” “2,” “1”) 66 may occur. At the end of the countdown, indication 54 “Shocking Patient” reports that the automated control has applied the defibrillation shock to patient 46. Similarly, FIG. 12 shows semi-automatic operation of personal defibrillation device 16, as indicated by the “Patient heart rate and bodily signals warrant shock” report appearing on personal multifunction device 12. Then, at an appropriate time, the command “Input Shot Command” is made clear on personal multifunction device 12, directing the use to administer manually shock to patient 46 by pressing the “Press” button. Thereafter, “Shocking Patient” indication 54 appears on personal multifunction device 12.

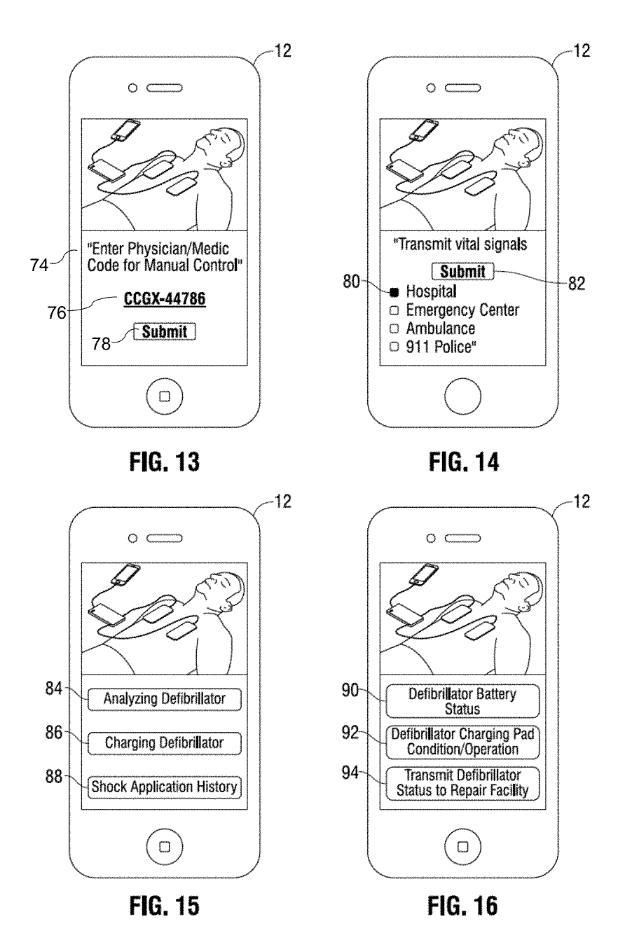

FIG. 13 displays an interface a physician or skilled medical professional may use to manually operate the portable defibrillator of the present disclosure. Thus, FIG. 13 illustrates aspects of a manual mode of control for portable defibrillation system 10 of the present disclosure. Thus, on personal multifunction device 12 an input screen with the direction “Enter Physician/Medic Code for Manual Control” provides the ability to insert a code, here “CCGX-44786,” at field 76, that may be provided to a physician or trained medic. By providing this input, the skilled person has the ability to assume totally manual control of portable defibrillation device 12 to apply therapeutic shock as his professional judgment may dictate.

FIG. 14 portrays the transmission of patient vital statistics from the presently disclosed portable defibrillation system 10 to a medical facility. Personal multifunction device 12 works in tandem with portable defibrillation device 16 to provide vital information that may be collected, preprocessed, and then transmitted to a set 80 of emergency care centers such a hospital, an emergency center, an ambulance, and/or 911 police service that may provide further care for patient 46.

FIGS. 15 and 16 show portable defibrillation system 10 reports include self-monitoring reports that defibrillation software app 14 may provide. Thus, on portable multifunction device 12 an analyzing defibrillator function 84 appears at analyzing defibrillator icon 70 to indicate using defibrillation application 14 to assess the status of portable defibrillator device 16. Indicator 86 reports that portable defibrillator device 16 is charging pads 20 and 22. Also, shock application history icon 88 may access the history of shock application for both presentation and analysis by the user.

FIG. 16 displays additional functions that defibrillation application 14 provides concerning the status of portable defibrillator device 16. Thus, by selecting button 90 the user may access defibrillator battery status functions. Selectable button 92 indicates that condition of defibrillator charging pads 20 and 22, as to their present condition or their being operated. And, importantly, button 94 indicates to the user that the portable defibrillator device 16 needs repair and should be transferred to repair facility.

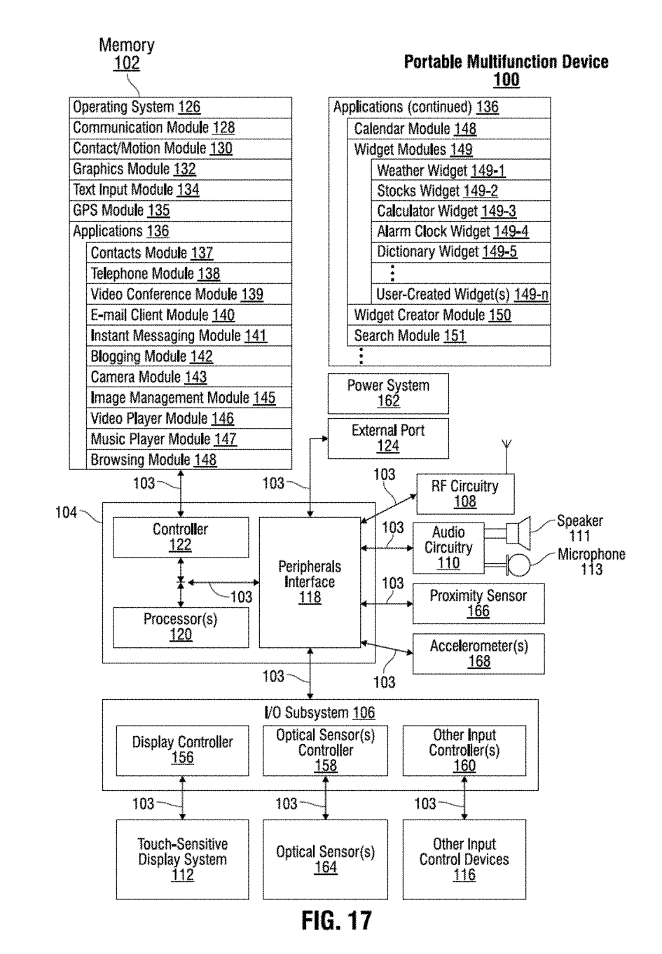

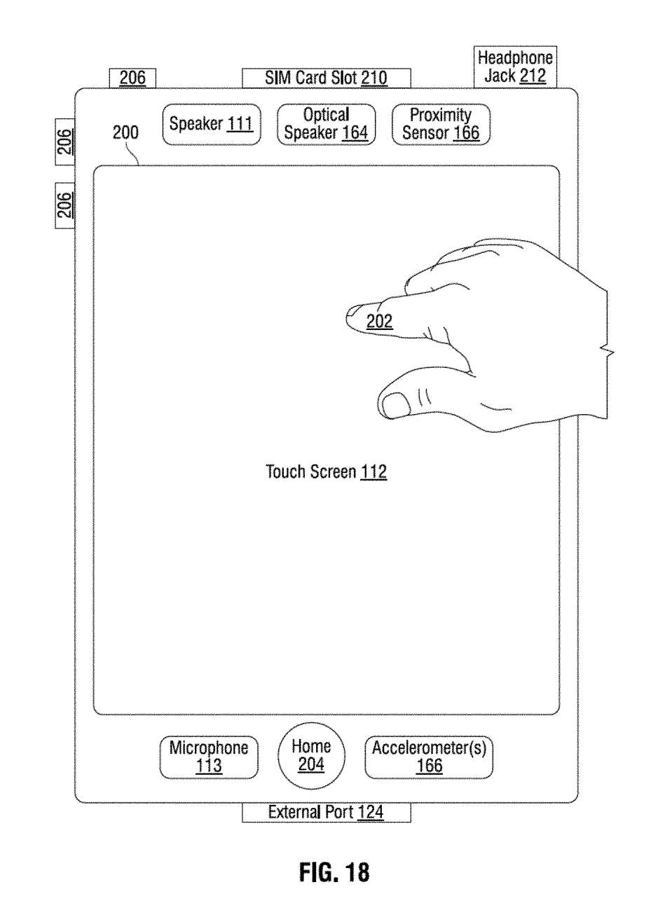

FIGS. 17 and 18 show an exemplary personal multifunction device for use with the portable defibrillator of the present disclosure. FIG. 17 shows block diagrams illustrating portable multifunction devices 100 with touch-sensitive displays 112 in accordance with some embodiments for the presently disclosed method, system, and portable defibrillation system 10 for delivering a dose of electric current or counter shock to a heart, for depolarizing a large amount of the heart muscle, thereby ending the dysrhythmia and in response thereto allowing the heart to re-establish a normal sinus rhythm. The touch-sensitive display 112 is sometimes called a “touch screen” for convenience, and may also be known as or called a touch-sensitive display system. The portable multifunction device 100 may include a memory 102 (which may include one or more computer readable storage mediums), a memory controller 122, one or more processing units (CPU's) 120, a peripherals interface 118, RF circuitry 108, audio circuitry 110, a speaker 111, a microphone 113, an input/output (I/O) subsystem 106, other input or control devices 116, and an external port 124. The portable multifunction device 100 may include one or more optical sensors 164. These components may communicate over one or more communication buses or signal lines 103.

It should be appreciated that the portable multifunction device 100 is only one example of a portable multifunction device 100, and that the portable multifunction device 100 may have more or fewer components than shown, may combine two or more components, or a may have a different configuration or arrangement of the components. The various components shown in FIG. 17 may be implemented in hardware, software or a combination of both hardware and software, including one or more signal processing and/or application specific integrated circuits.

Memory 102 may include high-speed random access memory and may also include non-volatile memory, such as one or more magnetic disk storage devices, flash memory devices, or other non-volatile solid-state memory devices. Access to memory 102 by other components of the portable multifunction device 100, such as the CPU 120 and the peripherals interface 118, may be controlled by the memory controller 122.

The peripherals interface 118 couples the input and output peripherals of the device to the CPU 120 and memory 102. The one or more processors 120 run or execute various software programs and/or sets of instructions stored in memory 102 to perform various functions for the portable multifunction device 100 and to process data.

In some embodiments, the peripherals interface 118, the CPU 120, and the memory controller 122 may be implemented on a single chip, such as a chip 104. In some other embodiments, they may be implemented on separate chips.

The RF (radio frequency) circuitry 108 receives and sends RF signals, also called electromagnetic signals. The RF circuitry 108 converts electrical signals to/from electromagnetic signals and communicates with communications networks and other communications devices via the electromagnetic signals. The RF circuitry 108 may include well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

The RF circuitry 108 may communicate with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The wireless communication may use any of a plurality of communications standards, protocols and technologies, including but not limited to Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.1 in), voice over Internet Protocol (VoIP), Wi-MAX, a protocol for email (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), and/or Instant Messaging and Presence Service (IMPS)), and/or Short Message Service (SMS)), or any other suitable communication protocol, including communication protocols not yet developed as of the filing date of this document.

The audio circuitry 110, the speaker 111, and the microphone 113 provide an audio interface between a user and the portable multifunction device 100. The audio circuitry 110 receives audio data from the peripherals interface 118, converts the audio data to an electrical signal, and transmits the electrical signal to the speaker 111. The speaker 111 converts the electrical signal to human-audible sound waves. The audio circuitry 110 also receives electrical signals converted by the microphone 113 from sound waves. The audio circuitry 110 converts the electrical signal to audio data and transmits the audio data to the peripherals interface 118 for processing. Audio data may be retrieved from and/or transmitted to memory 102 and/or the RF circuitry 108 by the peripherals interface 118. In some embodiments, the audio circuitry 110 also includes a headset jack (e.g. 212, FIG. 18). The headset jack provides an interface between the audio circuitry 110 and removable audio input/output peripherals, such as output-only headphones or a headset with both output (e.g., a headphone for one or both ears) and input (e.g., a microphone).

The I/O subsystem 106 couples input/output peripherals on the portable multifunction device 100, such as the touch screen 112 and other input/control devices 116, to the peripherals interface 118. The I/O subsystem 106 may include a display controller 156 and one or more input controllers 160 for other input or control devices. The one or more input controllers 160 receive/send electrical signals from/to other input or control devices 116. The other input/control devices 116 may include physical buttons (e.g., push buttons, rocker buttons, etc.), dials, slider switches, joysticks, click wheels, and so forth. In some alternate embodiments, input controller(s) 160 may be coupled to any (or none) of the following: a keyboard, infrared port, USB port, and a pointer device such as a mouse. The one or more buttons (e.g., 208, FIG. 18) may include an up/down button for volume control of the speaker 111 and/or the microphone 113. The one or more buttons may include a push button (e.g., 206, FIG. 18). A quick press of the push button may disengage a lock of the touch screen 112 or begin a process that uses gestures on the touch screen to unlock the device, as described in U.S. patent application Ser. No. 11/322,549, “Unlocking a Device by Performing Gestures on an Unlock Image,” filed Dec. 23, 2005, which is hereby incorporated by reference in its entirety. A longer press of the push button (e.g., 206) may turn power to the portable multifunction device 100 on or off. The user may be able to customize a functionality of one or more of the buttons. The touch screen 112 is used to implement virtual or soft buttons and one or more soft keyboards.

The touch-sensitive touch screen 112 provides an input interface and an output interface between the device and a user. The display controller 156 receives and/or sends electrical signals from/to the touch screen 112. The touch screen 112 displays visual output to the user. The visual output may include graphics, text, icons, video, and any combination thereof (collectively termed “graphics”). In some embodiments, some or all the visual output may correspond to user-interface objects, further details of which are described below.

A touch screen 112 has a touch-sensitive surface, sensor or set of sensors that accepts input from the user based on haptic and/or tactile contact. The touch screen 112 and the display controller 156 (along with any associated modules and/or sets of instructions in memory 102) detect contact (and any movement or breaking of the contact) on the touch screen 112 and converts the detected contact into interaction with user-interface objects (e.g., one or more soft keys, icons, web pages or images) that are displayed on the touch screen. In an exemplary embodiment, a point of contact between a touch screen 112 and the user corresponds to a finger of the user.

The touch screen 112 may use LCD (liquid crystal display) technology, or LPD (light emitting polymer display) technology, although other display technologies may be used in other embodiments. The touch screen 112 and the display controller 156 may detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with a touch screen 112.

A touch-sensitive display in some embodiments of the touch screen 112 may be analogous to the multi-touch sensitive tablets described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in their entirety. However, a touch screen 112 displays visual output from the portable multifunction device 100, whereas touch sensitive tablets do not provide visual output.

A touch-sensitive display in some embodiments of the touch screen 112 may be as described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures for Touch Sensitive Input Devices,” filed Jul. 30, 2004; (4) U.S. patent application Ser. No. 11/048,264, “Gestures for Touch Sensitive Input Devices,” filed Jan. 31, 2005; (5) U.S. patent application Ser. No. 11/038,590, “Mode-Based Graphical User Interfaces for Touch Sensitive Input Devices,” filed Jan. 18, 2005; (6) U.S. patent application Ser. No. 11/228,758, “Virtual Input Device Placement on A Touch Screen User Interface,” filed Sep. 16, 2005; (7) U.S. patent application Ser. No. 11/228,700, “Operation of A Computer with A Touch Screen Interface,” filed Sep. 16, 2005; (8) U.S. patent application Ser. No. 11/228,737, “Activating Virtual Keys of a Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

The touch screen 112 may have a resolution in excess of 100 dpi. In an exemplary embodiment, the touch screen has a resolution of approximately 160 dpi. The user may contact the touch screen 112 using any suitable object or appendage, such as a stylus, a finger, and so forth. In some embodiments, the user interface is designed to work primarily with finger-based contacts and gestures, which are much less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, the portable multifunction device 100 may include a touchpad (not shown) for activating or deactivating functions. In some embodiments, the touchpad is a touch-sensitive area of the device that, unlike the touch screen, does not display visual output. The touchpad may be a touch-sensitive surface that is separate from the touch screen 112 or an extension of the touch-sensitive surface formed by the touch screen.

The portable multifunction device 100 also includes a power system 162 for powering the various components. The power system 162 may include a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

The portable multifunction device 100 may also include one or more optical sensors 164. FIG. 17 shows an optical sensor coupled to an optical sensor controller 158 in I/O subsystem 106. The optical sensor 164 may include charge-coupled device (CCD) or complementary metal-oxide semiconductor (CMOS) phototransistors. The optical sensor 164 receives light from the environment, projected through one or more lens, and converts the light to data representing an image. In conjunction with an imaging module 143 (also called a camera module), the optical sensor 164 may capture still images or video. In some embodiments, an optical sensor is located on the back of the portable multifunction device 100, opposite the touch screen display 112 on the front of the device, so that the touch screen display may be used as a viewfinder for either still and/or video image acquisition.

In some embodiments, an optical sensor is located on the front of the device so that the user's image may be obtained for videoconferencing while the user views the other video conference participants on the touch screen display. In some embodiments, the position of the optical sensor 164 can be changed by the user (e.g., by rotating the lens and the sensor in the device housing) so that a single optical sensor 164 may be used along with the touch screen display for both video conferencing and still and/or video image acquisition.

The portable multifunction device 100 may also include one or more proximity sensors 166. FIG. 17 show a proximity sensor 166 coupled to the peripherals interface 118. Alternately, the proximity sensor 166 may be coupled to an input controller 160 in the I/O subsystem 106. The proximity sensor 166 may perform as described in U.S. patent application Ser. No. 11/241,839, “Proximity Detector in Handheld Device,” filed Sep. 30, 2005; Ser. No. 11/240,788, “Proximity Detector in Handheld Device,” filed Sep. 30, 2005; Ser. No. 11/620,702, “Using Ambient Light Sensor to Augment Proximity Sensor Output”; Ser. No. 11/586,862, “Automated Response to And Sensing of User Activity in Portable Devices,” filed Oct. 24, 2006; and Ser. No. 11/638,251, “Methods and Systems for Automatic Configuration of Peripherals,” which are hereby incorporated by reference in their entirety. In some embodiments, the proximity sensor turns off and disables the touch screen 112 when the multifunction device is placed near the user's ear (e.g., when the user is making a phone call). In some embodiments, the proximity sensor keeps the screen off when the device is in the user's pocket, purse, or other dark area to prevent unnecessary battery drainage when the device is a locked state.

The portable multifunction device 100 may also include one or more accelerometers 168. FIG. 17 shows an accelerometer 168 coupled to the peripherals interface 118. Alternately, the accelerometer 168 may be coupled to an input controller 160 in the I/O subsystem 106. The accelerometer 168 may perform as described in U.S. Patent Publication No. 20050190059, “Acceleration-based Theft Detection System for Portable multifunction devices,” and U.S. Patent Publication No. 20060017692, “Methods and Apparatuses for Operating a Portable Device Based on An Accelerometer,” both of which are which are incorporated by reference in their entirety. In some embodiments, information is displayed on the touch screen display in a portrait view or a landscape view based on an analysis of data received from the one or more accelerometers.

In some embodiments, the software components stored in memory 102 may include an operating system 126, a communication module (or set of instructions) 128, a contact/motion module (or set of instructions) 130, a graphics module (or set of instructions) 132, a text input module (or set of instructions) 134, a Global Positioning System (GPS) module (or set of instructions) 135, and applications (or set of instructions) 136.

The operating system 126 (e.g., Darwin, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

The communication module 128 facilitates communication with other devices over one or more external ports 124 and also includes various software components for handling data received by the RF circuitry 108 and/or the external port 124. The external port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin or lightning) connector that is the same as, or similar to and/or compatible with the 30-pin or lightning connector used on iPod (trademark of Apple Computer, Inc.) devices.

The contact/motion module 130 may detect contact with the touch screen 112 (in conjunction with the display controller 156) and other touch sensitive devices (e.g., a touchpad or physical click wheel). The contact/motion module 130 includes various software components for performing various operations related to detection of contact, such as determining if contact has occurred, determining if there is movement of the contact and tracking the movement across the touch screen 112, and determining if the contact has been broken (i.e., if the contact has ceased). Determining movement of the point of contact may include determining speed (magnitude), velocity (magnitude and direction), and/or an acceleration (a change in magnitude and/or direction) of the point of contact. These operations may be applied to single contacts (e.g., one finger contacts) or to multiple simultaneous contacts (e.g., “multitouch”/multiple finger contacts). In some embodiments, the contact/motion module 130 and the display controller 156 also detects contact on a touchpad. In some embodiments, the contact/motion module 130 and the controller 160 detects contact on a click wheel.

The graphics module 132 includes various known software components for rendering and displaying graphics on the touch screen 112, including components for changing the intensity of graphics that are displayed. As used herein, the term “graphics” includes any object that can be displayed to a user, including without limitation text, web pages, icons (such as user-interface objects including soft keys), digital images, videos, animations and the like. An animation in this context is a display of a sequence of images that gives the appearance of movement, and informs the user of an action that has been performed (such as moving an email message to a folder). In this context, a respective animation that confirms an action by the user of the device typically takes a predefined, finite amount of time, such as an amount of time between 0.2 and 1.0 seconds, or between 0.5 and 2.0 seconds, depending on the context.

The text input module 134, which may be a component of graphics module 132, provides soft keyboards for entering text in various applications (e.g., contacts 137, e-mail 140, IM 141, blogging 142, browser 147, and any other application that needs text input).

The GPS module 135 determines the location of the device and provides this information for use in various applications (e.g., to telephone 138 for use in location-based dialing, to camera 143 and/or blogger 142 as picture/video metadata, and to applications that provide location-based services such as weather widgets, local yellow page widgets, and map/navigation widgets).

The applications 136 may include the following modules (or sets of instructions), or a subset or superset thereof:
a contacts module 137 (sometimes called an address book or contact list);
a telephone module 138;
a video conferencing module 139;
an e-mail client module 140;
an instant messaging (IM) module 141;
a blogging module 142;
a camera module 143 for still and/or video images;
an image management module 144;
a video player module 145;
a music player module 146;
a browser module 147;
a calendar module 148;
widget modules 149, which may include
   weather widget 149-1,
   stocks widget 149-2,
   calculator widget 149-3,
   alarm clock widget 149-4,
   dictionary widget 149-5, and
   other widgets obtained by the user, as well as user-created widgets 149-6;
widget creator module 150 for making user-created widgets 149-6;
search module 151;
video and music player module 152, which merges video player module 145 and music player module 146;
notes module 153; and/or
map module 154.

Examples of other applications 136 that may be stored in memory 102 include other word processing applications, JAVA-enabled applications, encryption, digital rights management, voice recognition, and voice replication.

In conjunction with touch screen 112, display controller 156, contact module 130, graphics module 132, and text input module 134, the contacts module 137 may be used to manage an address book or contact list, including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone 138, video conference 139, e-mail 140, or IM 141; and so forth. Embodiments of user interfaces and associated processes using contacts module 137 are described further below.

In conjunction with RF circuitry 108, audio circuitry 110, speaker 111, microphone 113, touch screen 112, display controller 156, contact module 130, graphics module 132, and text input module 134, the telephone module 138 may be used to enter a sequence of characters corresponding to a telephone number, access one or more telephone numbers in the address book 137, modify a telephone number that has been entered, dial a respective telephone number, conduct a conversation and disconnect or hang up when the conversation is completed. As noted above, the wireless communication may use any of a plurality of communications standards, protocols and technologies. Embodiments of user interfaces and associated processes using telephone module 138 are described further below.

In conjunction with RF circuitry 108, audio circuitry 110, speaker 111, microphone 113, touch screen 112, display controller 156, optical sensor 164, optical sensor controller 158, contact module 130, graphics module 132, text input module 134, contact list 137, and telephone module 138, the videoconferencing module 139 may be used to initiate, conduct, and terminate a video conference between a user and one or more other participants.

In conjunction with RF circuitry 108, touch screen 112, display controller 156, contact module 130, graphics module 132, and text input module 134, the e-mail client module 140 may be used to create, send, receive, and manage e-mail. In conjunction with image management module 144, the e-mail module 140 makes it very easy to create and send e-mails with still or video images taken with camera module 143. Embodiments of user interfaces and associated processes using e-mail module 140 are described further below.

In conjunction with RF circuitry 108, touch screen 112, display controller 156, contact module 130, graphics module 132, and text input module 134, the instant messaging module 141 may be used to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages and to view received instant messages. In some embodiments, transmitted and/or received instant messages may include graphics, photos, audio files, video files and/or other attachments as are supported in a MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS). Embodiments of user interfaces and associated processes using instant messaging module 141 are described further below.

In conjunction with RF circuitry 108, touch screen 112, display controller 156, contact module 130, graphics module 132, text input module 134, image management module 144, and browsing module 147, the blogging module 142 may be used to send text, still images, video, and/or other graphics to a blog (e.g., the user's blog).

In conjunction with touch screen 112, display controller 156, optical sensor(s) 164, optical sensor controller 158, contact module 130, graphics module 132, and image management module 144, the camera module 143 may be used to capture still images or video (including a video stream) and store them into memory 102, modify characteristics of a still image or video, or delete a still image or video from memory 102. Embodiments of user interfaces and associated processes using camera module 143 are described further below.

In conjunction with touch screen 112, display controller 156, contact module 130, graphics module 132, text input module 134, and camera module 143, the image management module 144 may be used to arrange, modify or otherwise manipulate, label, delete, present (e.g., in a digital slide show or album), and store still and/or video images. Embodiments of user interfaces and associated processes using image management module 144 are described further below.

In conjunction with touch screen 112, display controller 156, contact module 130, graphics module 132, audio circuitry 110, and speaker 111, the video player module 145 may be used to display, present or otherwise play back videos (e.g., on the touch screen or on an external, connected display via external port 124). Embodiments of user interfaces and associated processes using video player module 145 are described further below.

In conjunction with touch screen 112, display system controller 156, contact module 130, graphics module 132, audio circuitry 110, speaker 111, RF circuitry 108, and browser module 147, the music player module 146 allows the user to download and play back recorded music and other sound files stored in one or more file formats, such as MP3 or AAC files. In some embodiments, the portable multifunction device 100 may include the functionality of an MP3 player, such as an iPod (trademark of Apple Computer, Inc.). Embodiments of user interfaces and associated processes using music player module 146 are described further below.

In conjunction with RF circuitry 108, touch screen 112, display system controller 156, contact module 130, graphics module 132, and text input module 134, the browser module 147 may be used to browse the Internet, including searching, linking to, receiving, and displaying web pages or portions thereof, as well as attachments and other files linked to web pages. Embodiments of user interfaces and associated processes using browser module 147 are described further below.

In conjunction with RF circuitry 108, touch screen 112, display system controller 156, contact module 130, graphics module 132, text input module 134, e-mail module 140, and browser module 147, the calendar module 148 may be used to create, display, modify, and store calendars and data associated with calendars (e.g., calendar entries, to do lists, etc.). Embodiments of user interfaces and associated processes using calendar module 148 are described further below.

In conjunction with RF circuitry 108, touch screen 112, display system controller 156, contact module 130, graphics module 132, text input module 134, and browser module 147, the widget modules 149 are mini-applications that may be downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display system controller 156, contact module 130, graphics module 132, text input module 134, and browser module 147, the widget creator module 150 may be used by a user to create widgets (e.g., turning a user-specified portion of a web page into a widget).

In conjunction with touch screen 112, display system controller 156, contact module 130, graphics module 132, and text input module 134, the search module 151 may be used to search for text, music, sound, image, video, and/or other files in memory 102 that match one or more search criteria (e.g., one or more user-specified search terms).

In conjunction with touch screen 112, display controller 156, contact module 130, graphics module 132, and text input module 134, the notes module 153 may be used to create and manage notes, to do lists, and the like.

In conjunction with RF circuitry 108, touch screen 112, display system controller 156, contact module 130, graphics module 132, text input module 134, GPS module 135, and browser module 147, the map module 154 may be used to receive, display, modify, and store maps and data associated with maps (e.g., driving directions; data on stores and other points of interest at or near a particular location; and other location-based data).

Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. For example, video player module 145 may be combined with music player module 146 into a single module (e.g., video and music player module 152, FIG. 17). In some embodiments, memory 102 may store a subset of the modules and data structures identified above. Furthermore, memory 102 may store additional modules and data structures not described above.

In some embodiments, the portable multifunction device 100 is a device where operation of a predefined set of functions on the device is performed exclusively through a touch screen 112 and/or a touchpad. By using a touch screen and/or a touchpad as the primary input/control device for operation of the portable multifunction device 100, the number of physical input/control devices (such as push buttons, dials, and the like) on the portable multifunction device 100 may be reduced.

The predefined set of functions that may be performed exclusively through a touch screen and/or a touchpad include navigation between user interfaces. In some embodiments, the touchpad, when touched by the user, navigates the portable multifunction device 100 to a main, home, or root menu from any user interface that may be displayed on the portable multifunction device 100. In such embodiments, the touchpad may be referred to as a “menu button.” In some other embodiments, the menu button may be a physical push button or other physical input/control device instead of a touchpad.

FIG. 18 illustrates a portable multifunction device 100 having a touch screen 112 in accordance with some embodiments. The touch screen may display one or more graphics within user interface (UI) 200. In this embodiment, as well as others described below, a user may select one or more of the graphics by making contact or touching the graphics, for example, with one or more fingers 202 (not drawn to scale in the FIGURE). In some embodiments, selection of one or more graphics occurs when the user breaks contact with the one or more graphics. In some embodiments, the contact may include a gesture, such as one or more taps, one or more swipes (from left to right, right to left, upward and/or downward) and/or a rolling of a finger (from right to left, left to right, upward and/or downward) that has contacted the portable multifunction device 100. In some embodiments, inadvertent contact with a graphic may not select the graphic. For example, a swipe gesture that sweeps over a personal multifunction app icon may not select the corresponding application when the gesture corresponding to selection is a tap.

The portable multifunction device 100 may also include one or more physical buttons, such as “home” or menu button 204. As described previously, the menu button 204 may be used to navigate to any application 136 in a set of applications that may be executed on the portable multifunction device 100. Alternatively, in some embodiments, the menu button is implemented as a soft key in a GUI in touch screen 112.

In one embodiment, the portable multifunction device 100 includes a touch screen 112, a menu button 204, a push button 206 for powering the device on/off and locking the device, volume adjustment button(s) 208, a Subscriber Identity Module (SIM) card slot 210, a head set jack 212, and a docking/charging external port 124. The push button 206 may be used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, the portable multifunction device 100 also may accept verbal input for activation or deactivation of some functions through the microphone 113.

Additional embodiments of the present disclosure made advantageous use of (a) light coding technology, (b) three-dimensional sensing using speckle patterns, (c) depth-varying light fields for three dimensional sensing, (d) systems and methods for imaging and image processing for creating an image having blurred and non-blurred areas, (e) real-time camera tracking using depth maps, and (f) depth map calculation in a stereo camera system, all as may be applicable to the present disclosure and the advantages thereof.

FIGS. 19 and 20 present functional aspects of the system of the present disclosure. FIG. 19 illustrates a software logic 300 flow chart representing how a user might operate the exemplary embodiment in a non-emergency situation 310. A user would launch a personal multifunction app 310 on an external personal multifunction device, which may include a personal multifunction device 12. The application would generate a prompt asking if an emergency is occurring 312. If it is a non-emergency situation; the user would choose “non-emergency” 314. The application would then present a menu on the screen 316 allowing the user to select between options that may include: electro-cardiograph 318, training 332, videos 346, settings 454, and testing 460.

In the event the user selects electro-cardiograph 318, the user will be instructed to connect 320 the portable defibrillator device 16 to the personal multifunction device 12. The user will follow on screen instructions to properly position the pads 328. Once the pads are properly positioned, the user will remain still 330. The portable defibrillator device 16 will then begin importing and recording data 322. It may amplify the signal in hardware and transmit that data to the application on the personal multifunction device 12 or other external personal multifunction device 324. The application will then filter the input signal and display the results 326 on the screen of the personal multifunction device 12.

In the event the user selects training 332, the user will follow onscreen instructions to locate 334 the portable defibrillator device 16. A training scenario 336 will be presented on the screen of the personal multifunction device 12, and the user will follow instructions displayed on the screen about how to properly position the victim 338. The on-screen instructions will instruct the user on how to locate the pads and how to place them properly on the person suffering from a cardiac arrest 340. The application will then explain how the smart savior system works 342, and provide next steps and possible scenarios 344.

Responsive to selection of videos 346, health related educational videos will be presented on screen 348. The user may select a video and the application will then play the selected video and instructions accompanying the video 350. When the video is finished playing, the screen will return to the video options and present the health related educational video options 352.

In the event the user selects settings 354, the user will be presented with a screen to input their personal information and personal medical information including their medical history 356. Next, the screen will instruct the user to input emergency contact information 358.

In the event the user selects testing 360, the screen will instruct the user to connect 362 the portable defibrillator device 16 to the personal multifunction device 12. The connection will be automatically detected by the personal multifunction app 368. After the connection, the application will test the battery life 470 of the portable defibrillator device 16, and the charging circuit 472 or electrical system of the portable defibrillator device 16.

FIG. 20 illustrates a flow chart representing how a user might operate the portable defibrillation system 10 in an emergency 400. The user will recognize that an emergency is occurring, then launch the application on the personal multifunction device 12. The application will ask the user if it is an emergency 404. The user will select emergency 406. Portable defibrillation system 10 will instruct the user to properly position the patient 408 and to properly position the charging pads 410. Then portable defibrillation system 10 instructs the user to step away from the patient 412.

Portable defibrillation system 10 will signal the portable defibrillator device 16 to startup and charge the personal multifunction device 12 at 414. The personal multifunction device 12 is charged to prevent the personal multifunction device 12 from dying during operation which would mean the loss of functionality of the entire defibrillator system. The application will begin importing and recording data 416 from the portable defibrillator device 16. The portable defibrillator device 16 will amplify the signal and transmit the signal to the personal multifunction device 12 application 418. The application will then filter the incoming signal and display 420 the signal on the screen of the personal multifunction device 12.

Next, the application will analyze the heart rate data that is provided from the incoming signal 422. If the heart rate is normal or no shock is needed 432 the screen will display that the heartrate is normal and no shock is needed 434.

The application will continue analyzing the incoming signal and heart rate data for changes. If a shockable heartrate is recognized 424, the application will display on the screen of the personal multifunction device 12 that a shock is needed 426 and that charging is in progress 428. The application will then warn the user of the personal multifunction device 12 to clear away from the victim of the cardiac arrest 430.

The application will then control the portable defibrillator device 16 to initiate the first shock at a lower level of the settings. The application will stop the charging process 546 and discharge the shock 548 on the electrodes attached to the victim. The application will then stop the shock discharge 550. After the first shock 436, the application will re-analyze the heart rate data 422. If another charge is required, the application will initiate the electronic system of the portable defibrillator device 16 to charge to a higher level 446. When the appropriate level is reached, the application will stop the charging process 448 and initiate the discharge of the shock to the electrodes or charging pads 450 followed by a stop discharge signal 552. The application will then re-evaluate the heart rate data and repeat the charging, discharging process as needed.

In summary, here is disclosed a portable defibrillator device for use with a mobile phone or personal multifunction device for delivering a dose of electric current or counter shock to a heart. The disclosed subject matter provides the electric shock for depolarizing a large amount of the heart muscle, thereby ending dysrhythmia and in response thereto allowing the heart to re-establish a normal sinus rhythm. The disclosure includes a portable defibrillator case for containing defibrillation circuitry supporting the generation and transmission of a dose of electric current or counter shock. A set of charging pads electrically connect to the portable defibrillator case for communication of the dose of electric current or counter shock to a patient and sensing from the patient responses to said electric current or counter shock. A first set of communication circuitry associated with said portable defibrillator case for communicating between said defibrillation circuitry and said mobile phone or personal multifunction device. A second set of communication circuitry associated with said portable defibrillator case for communicating between said defibrillation circuitry and said set of charging pads. The portable defibrillator case, the set of charging pads, and the first and second sets of communication circuitry are of a size essentially similar to the size of the mobile phone or personal multifunction device.

The portable defibrillator device includes defibrillation circuitry having a charging circuit for generating and transmitting from said portable defibrillator case a defibrillation charge of electricity of sufficient charge and current for depolarizing at least a portion of a heart muscle for ending dysrhythmia in the heart muscle. A set of charging pad circuits receive the defibrillation charge and apply said defibrillation charge to the torso of a patient in the proximity of the heart and a set of charging pads comprising charging circuitry for applying a dose of electric current or counter shock to a heart in response to energization from said charging pad circuits.

The portable defibrillator further includes an EKC sensing circuit for generating, sending and receiving from said portable defibrillator case EKG sensing signals for monitoring the heart during the administration of said defibrillation charge. The set of charging pad circuits further comprise EKG sensing circuitry for sending and receiving EKG heart monitoring signals between said set of charging pads and said mobile phone or personal multifunction device. The set of charging pads comprise EKG signal circuitry for generating EKG sensing signals for transmission to said EKG sensing circuitry. The portable defibrillator device circuitry further includes a resistance sensing circuit for generating, sending and receiving from said portable defibrillator case a plurality of resistance sensing signals for monitoring the heart and operation of said EKG sensing circuit and said set of charging pads during the administration of said defibrillation charge. The resistance sensing circuit further comprises resistance reporting for reporting a plurality of resistance sensing signals for assessing and reporting the operational status of said portable defibrillator device.

The portable defibrillator device further includes interface circuitry for interfacing the portable defibrillator device with a mobile phone or personal multifunction device. This supports coordinating the operations and the portable defibrillator device with the mobile phone or personal multifunction device to provide controlled delivery of a dose of electric current or counter shock to a heart and continuous monitoring of the status of the heart in response to said controlled delivery.

The portable defibrillator circuitry includes a wireless communication circuit for communicating wireless communications with a plurality of communications circuits providing services related to the operation of said portable defibrillation device. A set of operational and sensing instructions for storing on a memory circuit associated with said mobile phone or personal multifunction device, said set of operational and sensing instructions for controlling the operation of said portable defibrillator device using the mobile phone or personal multifunction device.

The embodiments described above are exemplary and are not to be taken as limiting in any way. They are merely illustrative of the principles of the disclosure. Various changes, modifications and alternatives will be apparent to one skilled in the art. Accordingly, it is intended that the art disclosed shall be limited only to the extent required by the appended claims and the rules and principles of applicable law.

We claim:

1. A portable defibrillator device for use with a mobile phone or personal multifunction device for delivering a dose of electric current or countershock to a heart, for depolarizing a large amount of the heart muscle, thereby ending the dysrhythmia and in response thereto allowing the heart to re-establish a normal sinus rhythm, comprising:
a portable defibrillator case for containing defibrillation circuitry supporting the generation and transmission of a dose of electric current or countershock;
a set of charging pads electrically connected to said portable defibrillator case for communication of said dose of electric current or countershock to a patient and sensing from the patient responses to said electric current or countershock;
a first set of communication circuitry associated with said portable defibrillator case for communicating between said defibrillation circuitry and said mobile phone or personal multifunction device; and
a second set of communication circuitry associated with said portable defibrillator case for communicating between said defibrillation circuitry and said set of charging pads; and
wherein said portable defibrillator case, said set of charging pads, and said first and second sets of communication circuitry are of a size essentially similar to the size of the mobile phone or personal multifunction device.

2. The portable defibrillator device of claim 1, wherein said defibrillation circuitry comprises:
a charging circuit for generating and transmitting from said portable defibrillator case a defibrillation charge of electricity of sufficient charge and current for depolarizing at least a portion of a heart muscle for ending dysrhythmia in the heart muscle;
a set of charging pad circuits for receiving said defibrillation charge and applying said defibrillation charge to the torso of a patient in the proximity of the heart; and
a set of charging pads comprising charging circuitry for applying a dose of electric current or countershock to a heart in response to energization from said charging pad circuits.

3. The portable defibrillator device of claim 2, wherein said defibrillation circuitry further comprises:
an EKC sensing circuit for generating, sending and receiving from said portable defibrillator case EKG sensing signals for monitoring the heart during the administration of said defibrillation charge;
wherein said set of charging pad circuits further comprise EKG sensing circuitry for sending and receiving EKG heart monitoring signals between said set of charging pads and said mobile phone or personal multifunction device;
wherein said set of charging pads comprise EKG signal circuitry for generating EKG sensing signals for transmission to said EKG sensing circuitry.

4. The portable defibrillator device of claim 2, wherein said defibrillation circuitry further comprises:
a resistance sensing circuit for generating, sending and receiving from said portable defibrillator case a plurality of resistance sensing signals for monitoring the heart and operation of said EKG sensing circuit and said set of charging pads during the administration of said defibrillation charge; and
wherein said resistance sensing circuit further comprises resistance reporting for reporting a plurality of resistance sensing signals for assessing and reporting the operational status of said portable defibrillator device.

5. The portable defibrillator device of claim 1, further comprising interface circuitry for interfacing portable defibrillator device with a mobile phone or personal multifunction device, thereby coordinating the operations and said portable defibrillator device with the mobile phone or personal multifunction device to provide controlled delivery of a dose of electric current or countershock to a heart and continuous monitoring of the status of the heart in response to said controlled delivery.

6. The portable defibrillator device of claim 1, wherein said defibrillation circuitry comprises:
a wireless communication circuit for communicating wireless communications with a plurality of communications circuits providing services related to the operation of said portable defibrillation device.

7. The portable defibrillator device of claim 1, further comprising:
a set of operational and sensing instructions for storing on a memory circuit associated with said mobile phone or personal multifunction device, said set of operational and sensing instructions for controlling the operation of said portable defibrillator device using the mobile phone or personal multifunction device;
a charging circuit for generating and transmitting from said portable defibrillator case a defibrillation charge of electricity of sufficient charge and current for depolarizing at least a portion of a heart muscle for ending dysrhythmia in the heart muscle;
a set of charging pad circuits for receiving said defibrillation charge and applying said defibrillation charge to the torso of a patient in the proximity of the heart; and
a set of charging pads comprising charging circuitry for applying a dose of electric current or countershock to a heart in response to energization from said charging pad circuits.

8. A method for delivering a dose of electric current or counter shock using a heart portable defibrillator device with a mobile phone or personal multifunction device for depolarizing a large amount of the heart muscle, thereby ending the dysrhythmia and in response thereto allowing the heart to re-establish a normal sinus rhythm, comprising the steps of:
containing defibrillation circuitry supporting the generation and transmission of a dose of electric current or counter shock within a portable defibrillator case;
communicating said dose of electric current or counter shock to a patient using a set of charging pads electrically connected to said portable defibrillator case and sensing from the patient responses to said electric current or counter shock;
communicating between said defibrillation circuitry and said mobile phone or personal multifunction device using a first set of communication circuitry associated with said portable defibrillator case; and
communicating between said defibrillation circuitry and said set of charging pads using a second set of communication circuitry associated with said portable defibrillator case;
wherein said portable defibrillator case, said set of charging pads, and said first and second sets of communication circuitry are of a size essentially similar to the size of the mobile phone or personal multifunction device.

9. The method for delivering a dose of electric current or counter shock of claim 8, further comprising the steps of:
generating and transmitting from said portable defibrillator case a defibrillation charge of electricity of sufficient charge and current for depolarizing at least a portion of a heart muscle for ending dysrhythmia in the heart muscle using a charging circuit;
receiving said defibrillation charge and applying said defibrillation charge to the torso of a patient in the proximity of the heart using a set of charging pad circuits; and
applying a dose of electric current or counter shock to a heart in response to energization from said charging pad circuits using a set of charging pads associated with said charging circuitry.

10. The method for delivering a dose of electric current or counter shock of claim 8, further comprising the steps of:
generating, sending and receiving from said portable defibrillator case EKG sensing signals for monitoring the heart during the administration of said defibrillation charge using an EKG sensing circuit;
sending and receiving EKG heart monitoring signals between said set of charging pads and said mobile phone or personal multifunction device using said set of charging pad circuits; and
generating EKG sensing signals for transmission to said EKG sensing circuitry using said set of charging pads.

11. The method for delivering a dose of electric current or counter shock of claim 10, further comprising the steps of:
a resistance sensing circuit for generating, sending and receiving from said portable defibrillator case a plurality of resistance sensing signals for monitoring the heart and operation of said EKG sensing circuit and said set of charging pads during the administration of said defibrillation charge; and
wherein said resistance sensing circuit further comprises resistance reporting for reporting a plurality of resistance sensing signals for assessing and reporting the operational status of said portable defibrillator device.

12. The method for delivering a dose of electric current or counter shock of claim 8, further comprising the steps of interfacing portable defibrillator device with a mobile phone or personal multifunction device, thereby coordinating the operations and said portable defibrillator device with the mobile phone or personal multifunction device to provide controlled delivery of a dose of electric current or counter shock to a heart and continuous monitoring of the status of the heart in response to said controlled delivery.

13. The method for delivering a dose of electric current or counter shock of claim 8, further comprising the steps of communicating wireless communications with a plurality of communications circuits providing services related to the operation of said portable defibrillation device.

14. The method for delivering a dose of electric current or counter shock of claim 8, further comprising the steps of storing on a memory circuit associated with said mobile phone or personal multifunction device a set of operational and sensing instructions for controlling the operation of said portable defibrillator device using the mobile phone or personal multifunction device.

15. A portable defibrillator system, comprising:
a mobile phone or personal multifunction device for delivering a dose of electric current or counter shock to a heart, for depolarizing a large amount of the heart muscle, thereby ending the dysrhythmia and in response thereto allowing the heart to re-establish a normal sinus rhythm, and a portable defibrillator device, comprising:
a portable defibrillator case for containing defibrillation circuitry supporting the generation and transmission of a dose of electric current or counter shock;
a set of charging pads electrically connected to said portable defibrillator case for communication of said dose of electric current or counter shock to a patient and sensing from the patient responses to said electric current or counter shock;
a first set of communication circuitry associated with said portable defibrillator case for communicating between said defibrillation circuitry and said mobile phone or personal multifunction device; and
a second set of communication circuitry associated with said portable defibrillator case for communicating between said defibrillation circuitry and said set of charging pads; and
wherein said portable defibrillator case, said set of charging pads, and said first and second sets of communication circuitry are of a size essentially similar to the size of the mobile phone or personal multifunction device.

16. The portable defibrillator system of claim 15, wherein said defibrillation circuitry comprises:
a charging circuit for generating and transmitting from said portable defibrillator case a defibrillation charge of electricity of sufficient charge and current for depolarizing at least a portion of a heart muscle for ending dysrhythmia in the heart muscle;
a set of charging pad circuits for receiving said defibrillation charge and applying said defibrillation charge to the torso of a patient in the proximity of the heart; and
a set of charging pads comprising charging circuitry for applying a dose of electric current or counter shock to a heart in response to energization from said charging pad circuits.

17. The portable defibrillator system of claim 16, wherein said defibrillation circuitry further comprises:
an EKG sensing circuit for generating, sending and receiving from said portable defibrillator case EKG sensing signals for monitoring the heart during the administration of said defibrillation charge;
wherein said set of charging pad circuits further comprise EKG sensing circuitry for sending and receiving EKG heart monitoring signals between said set of charging pads and said mobile phone or personal multifunction device;
wherein said set of charging pads comprise EKG signal circuitry for generating EKG sensing signals for transmission to said EKG sensing circuitry.

18. The portable defibrillator system of claim 16, wherein said defibrillation circuitry further comprises:
a resistance sensing circuit for generating, sending and receiving from said portable defibrillator case a plurality of resistance sensing signals for monitoring the heart and operation of said EKG sensing circuit and said set of charging pads during the administration of said defibrillation charge; and
wherein said resistance sensing circuit further comprises resistance reporting for reporting a plurality of resistance sensing signals for assessing and reporting the operational status of said portable defibrillator device.

19. The portable defibrillator system of claim 15, further comprising interface circuitry for interfacing portable defibrillator device with a mobile phone or personal multifunction device, thereby coordinating the operations and said portable defibrillator device with the mobile phone or personal multifunction device to provide controlled delivery of a dose of electric current or counter shock to a heart and continuous monitoring of the status of the heart in response to said controlled delivery.

20. The portable defibrillator system of claim 15, wherein said defibrillation circuitry comprises:
a wireless communication circuit for communicating wireless communications with a plurality of communications circuits providing services related to the operation of said portable defibrillation device.
